(19) United States
(12) Patent Application Publication
(10) Pub. No.: US 2026/0345216 A1
(43) Pub. Date: Nov. 5, 2026

(54) PULSED FIELD ABLATION (PFA) THERAPY BASED ON SENSOR INPUT

(71) Applicant: St. Jude Medical, Cardiology Division, Inc., St. Paul, MN (US)

(72) Inventors: Dylan J. Harbaugh, Big Lake, MN (US); Isaac Remer, Richfield, MN (US)

(21) Appl. No.: 19/666,979

(22) Filed: May 4, 2026

Related U.S. Application Data
(60) Provisional application No. 63/800,083, filed on May 5, 2025.

(51) Int. Cl. A61B 18/00; A61B 34/00; A61B 34/10; A61B 90/00

(52) U.S. Cl. CPC A61B 18/00; A61B 34/10; A61B 34/25; A61B 90/06; A61B 2018/00351; A61B 2018/00642; A61B 2018/00666; A61B 2018/00702; A61B 2034/107; A61B 2090/065

(57) ABSTRACT

A medical device system includes a catheter with a catheter distal end configured to be positioned in contact with or proximity to patient tissue. The medical device system is configured to initiate a therapy routine to deliver pulsed field ablation (PFA) energy to the patient tissue. The medical device system is further configured to receive sensor input associated with the catheter distal end. The medical device system is further configured to inhibit delivery of the pulsed field ablation (PFA) energy based on comparing the sensor input to a threshold.

### CROSS-REFERENCE TO RELATED APPLICATIONS

This application claims the benefit of and priority to U.S. provisional application 63/800,083, titled “PULSED FIELD ABLATION (PFA) THERAPY BASED ON SENSOR INPUT”, filed May 5, 2025, the contents of which are incorporated by reference herein.

### TECHNICAL FIELD OF THE INVENTION

This invention relates generally to catheter devices that ablate patient tissue as a therapy, and more specifically to catheter devices that deliver pulsed field ablation (PFA) therapy.

### BACKGROUND

Catheters are used in some medical procedures to deliver therapy to a patient, for example to deliver electrical energy to ablate patient tissue to treat one or more conditions such as cardiac arrhythmia. Some catheters may be used to perform PFA ablation therapy which uses short high-intensity pulses of energy to ablate heart tissue via electroporation, unlike traditional RF ablation which ablates tissue using heat.

### SUMMARY

In some aspects, the techniques described herein relate to a medical device, including: a catheter distal end configured to be positioned in contact with or proximal to patient tissue within a heart muscle of a patient; and a processor configured to: initiate a therapy routine to deliver pulsed field ablation (PFA) energy to the patient tissue; receive sensor input associated with the catheter distal end, wherein the sensor input includes a position of the catheter distal end; compare the position of the catheter distal end to a restricted area of patient tissue surrounding at least one cardiac conduction structure of the heart muscle; and inhibit delivery of the pulsed field ablation (PFA) energy responsive to the catheter distal end entering the restricted area of patient tissue surrounding the at least one cardiac conduction structure.

The at least one cardiac conduction structure may include at least one of a sinoatrial (SA) node, an atrioventricular (AV) node, or a bundle of His.

The restricted area of patient tissue may be selectable by an operator.

The processor may be further configured to: present an image with a proposed restricted area surrounding the at least one cardiac conduction structure to an operator via a display; and receive user input increasing or decreasing the restricted area relative to the proposed restricted area.

The processor may be further configured to: compare the position of the catheter distal end to an approved therapy area of patient tissue; and inhibit further delivery of the PFA energy responsive to the catheter distal end exiting the approved therapy area.

The approved therapy area may be positioned between a plurality of restricted areas, each restricted area surrounding a respective cardiac conduction structure.

The sensor input may further include an orientation of the catheter distal end.

The catheter distal end may include at least one of an electrode or a magnetic sensor configured to generate localization data representing the position of the catheter distal end.

In some aspects, the techniques described herein relate to a medical device, including: a catheter distal end configured to be positioned in contact with patient tissue; and a processor configured to: initiate a therapy routine to deliver pulsed field ablation (PFA) energy to the patient tissue; receive sensor input associated with the catheter distal end, wherein the sensor input includes one or more measured forces associated with the catheter distal end; and inhibit delivery of the pulsed field ablation (PFA) energy based on comparing the one or more measured forces to a threshold indicative of movement of the catheter distal end from an intended therapy position.

The sensor input may be associated with a contact force at a tip of the catheter distal end, and the threshold corresponds to a change in one or more of amplitude and direction of the contact force.

The catheter distal end may include a contact force sensor configured to measure a magnitude and direction of force upon the tip.

The contact force sensor may include at least three fiber optic gaps configured to be measured to determine the magnitude and direction of the contact force.

The sensor input may be associated with movement of the catheter distal end, and the threshold corresponds to a movement of the catheter distal end in one or more of an X, Y, or Z direction.

The catheter distal end may include an accelerometer configured to detect forces associated with movement of the catheter distal end along three orthogonal axes.

The threshold may be selectable by an operator.

In some aspects, the techniques described herein relate to a method, including: initiating a therapy routine to deliver pulsed field ablation (PFA) energy to patient tissue within a heart muscle of a patient; receiving sensor input associated with a catheter distal end positioned within the heart muscle, wherein the sensor input includes a position of the catheter distal end; comparing the position of the catheter distal end to a designated area of patient tissue associated with at least one cardiac conduction structure of the heart muscle; and inhibiting delivery of the pulsed field ablation (PFA) energy based on the comparing.

The designated area may be a restricted area of patient tissue surrounding the at least one cardiac conduction structure, and inhibiting delivery of the PFA energy includes inhibiting further delivery of the PFA energy responsive to the catheter distal end entering the restricted area of patient tissue.

The at least one cardiac conduction structure may include at least one of a sinoatrial (SA) node, an atrioventricular (AV) node, or a bundle of His.

The designated area may be an approved therapy area of patient tissue, and inhibiting delivery of the PFA energy includes inhibiting further delivery of the PFA energy responsive to the catheter distal end exiting the approved therapy area.

The method may further include: presenting an image with a proposed designated area of the heart muscle to an operator via a display; and receiving user input increasing or decreasing the designated area relative to the proposed designated area.

### BRIEF DESCRIPTION OF THE FIGURES

FIG. 1 is a diagram depicting one example of a medical device system configured to deliver PFA therapy and inhibit delivery of PFA therapy via a catheter based on sensor inputs.

FIG. 2 is a block diagram depicting one example of a medical device system configured to deliver PFA therapy and to inhibit delivery of PFA therapy via a catheter based on sensor input.

FIG. 3A depicts one example a two dimensional (2D) cross-sectional image of a cardiac muscle and a restricted area of patient tissue.

FIG. 3B is a diagram that depicts one example of a 3D image of a posterior view of the left atrium looking at the pulmonary veins of a cardiac muscle and a restricted area of patient tissue.

FIG. 4A depicts one example of a 2D cross-section image of a human cardiac muscle and an approved area of patient tissue.

FIG. 4B is a diagram that depicts one example of a 3D image of a posterior view of the left atrium looking at the pulmonary veins of a cardiac muscle and an approved area of patient tissue.

FIG. 5A is a diagram that depicts one example a 2D cross-sectional image of a human cardiac muscle and an approved area of patient tissue.

FIG. 5B is a diagram that depicts one example of a 3D image of a posterior view of the left atrium looking at the pulmonary veins of a cardiac muscle , a restricted area of patient tissue, and an approved area of patient tissue.

FIG. 6A is a diagram that depicts one example a 2D cross-sectional image of a human cardiac muscle, an initial starting point for the delivery of PFA, and a distance D from the initial starting point.

FIG. 6B is a diagram that depicts one example of a 3D image of a posterior view of the left atrium looking at the pulmonary veins of a cardiac muscle an initial starting point for the delivery of PFA, and a distance D from the initial starting point.

FIGS. 7A-7B depict one example of a catheter with a catheter distal end that includes a contact force sensor to measure force.

FIGS. 8A and 8B depict one example of a catheter with a catheter distal end that includes an accelerometer sensor configured to detect forces associated with movement of the catheter distal end.

FIG. 9 is a flow diagram that depicts one example of a method of operating a medical device system including a catheter configured to deliver PFA energy as part of a therapy routine.

### DETAILED DESCRIPTION

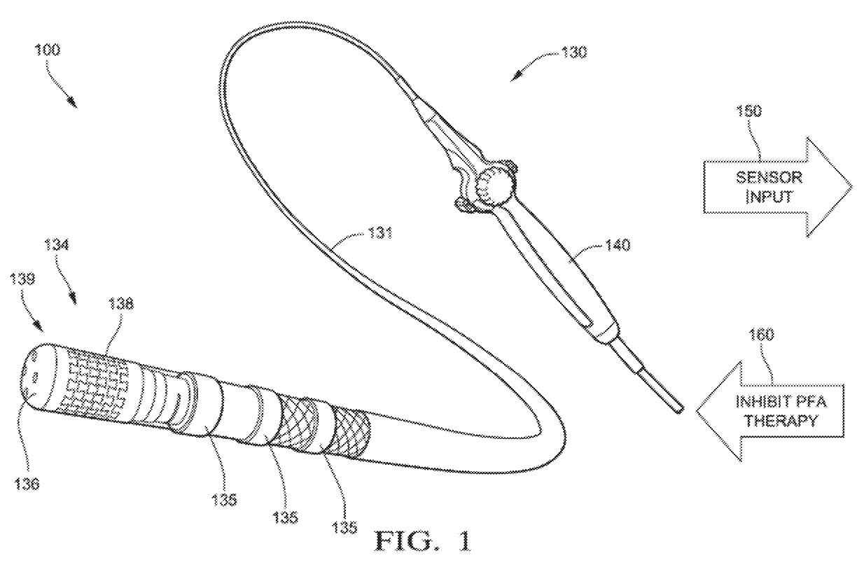

FIG. 1 is a diagram depicting one example of a medical device system 100 configured to deliver PFA therapy and selectively inhibit delivery of PFA therapy via a catheter 130. As shown in FIG. 1, the catheter 130 includes a handle 140, and a shaft 131, and a catheter distal end 134. The catheter distal end 134 is configured to be introduced into the body of a patient, specifically in the cardiovascular system in a patient and arranged in contact with and/or proximity to patient tissue and used to perform measurements and/or deliver therapy to a patient. In some examples, the catheter distal end 134 includes one or more sensor(s) used to measure patient characteristics. For example, the catheter distal end 134 may, as depicted in FIG. 1, include one or more electrodes, including ring electrodes 135 and/or a tip electrode 136 that may be used to perform measurements and/or to deliver electrical energy to patient tissue to deliver therapy such as ablation. As shown in FIG. 1, the ring electrodes 135 are arranged along a length of the catheter distal end 134. In the example of FIG. 1, the tip electrode 136 is an electrically conductive surface of a tip 139 of the catheter distal end 134, although more than one electrically isolated electrode may be arranged at the tip 139 in other examples.

In some examples, each of the ring electrodes 135 and/or tip electrodes 139 are electrically isolated from one another and/or other conductive structures of catheter distal end 134. For example, the ring electrodes 135 presented along a length of the catheter distal end 134 may be electrically isolated from one another, as well as from the tip electrode 136, such that each of the respective electrodes 135, 136 may be controlled to measure characteristics of patient tissue and or delivery therapy to a patient. For example, each of the respective electrodes 135, 136 shown in FIG. 1 may be used to measure tissue impedance, determine contact with patient tissue, and/or to operate as an anode or cathode to delivery therapy to patient tissue. In some examples, the catheter 130 depicted in FIG. 1 may be configured to deliver pulsed field ablation therapy via one or more of the electrodes 135, 136, which uses electrical pulses to ablate patient tissue to treat conditions such as cardiac arrhythmia and/or to remove tumors. In some examples, PFA therapy delivered via the catheter 130 by causing relatively high-amplitude electrical pulses to be delivered to target tissue. In some examples, PFA therapy delivered via the catheter 130 ablates targeted tissue by causing irreversible electroporation of the targeted tissue.

As mentioned above, one or more of the electrodes 135 and/or 136 may be used to perform measurements, specifically of patient characteristics, in addition or instead of being used to deliver PFA energy for therapy purposes. For example, the electrodes 135 and/or 136 may be used to measure electrical currents that traverse patient tissue to evaluate tissue health and/or the need for ablation therapy such as PFA. As another example, the electrodes may be used to measure an impedance of patient tissue, for example using bipolar electrode complex impedance (BECI), three-terminal impedance, two-terminal impedance, tetrapolar electrode complex impedance (TECI), or any other technique for measuring tissue impedance, for example to assess lesion formation in patient tissue. In other examples, the electrodes 135 and/or 136 may be used to detect to measure an impedance caused by one or more externally generated electric fields to localize the catheter distal end 134, i.e., to determine a position and/or orientation of the catheter distal end 134 in three-dimensional space.

In some examples, the catheter 130 includes one or more sensors other than the electrode(s) depicted in FIG. 1. For example, the catheter 130 may include one or more magnetic sensor(s), which may include one or more magnetic coil(s) of the catheter distal end 134 that are to localize the catheter distal end 134. Such magnetic coil(s) may be wrapped around a core material in some examples and/or be printed on a substrate material in other examples. According to these examples, the magnetic coil(s) may generate a measurable electrical signal responsive to one or more externally generated magnetic fields to localize the catheter distal end 134 in 3D space. Such magnetic localization data may be used alone or in combination with localization data from the electrode(s) 135 and/or 136 to localize the catheter distal end 134. According to examples where the electrode(s) 135/136 and/or magnetic sensor(s) are used to localize the catheter distal end 134, a determined position/orientation of the catheter distal end 134 may be used generate images presented to a physician/operator of the catheter 130 superimposed over images of patient tissue to help guide or otherwise manipulate the catheter 130.

In some examples, the catheter distal end 134 further includes sensor(s) configured to measure one or more forces associated with the catheter distal end 134, and the threshold(s) correspond to a rapid changes in force or force direction that indicate movement and/or slipping of the catheter distal end 134 from an intended location in contact with patient tissue. For example, the catheter distal end 134 may include one or more force sensor(s) such as one or more contact force sensors 138 configured to measure a contact force against the tip 139 of the catheter distal end 134, e.g., reflecting how forcefully the tip 139 is pressed against patient tissue and/or a direction of the force (e.g., whether the tip 139 is tilted relative to the patient tissue). For example, such a contact force sensor 138 may include three or more fiber optic “gaps” that may be monitored through white light Fabry-Perot interferometry to determine a magnitude and direction of a force upon the catheter distal end 134, for example by detect how much each respective gap is compressed. The catheter 130 may instead include other types of contact force sensors at the tip, including those that use fiber Bragg gratings (FBG), distributed optical fibers, a spring, piezoelectric element, or other flexible element to measure a contact force/orientation of the catheter distal end 134 against patient tissue. In still other examples, the catheter distal end 134 may include other force sensors such as an accelerometer configured to detect movement of the catheter distal end 134 along three orthogonal axes.

In the example of FIG. 1, the medical device system 100 is uniquely configured to deliver PFA therapy with heightened safety in comparison to traditional catheters by inhibiting the delivery PFA therapy 160 based on sensor input 150. For example, as shown in FIG. 1, the medical device system 100 may initiate a therapy routine to deliver pulsed field ablation (PFA) energy to patient tissue. The medical device system 100 is configured to receive sensor input 150 from one or more sensor(s) of associated with the catheter distal end 134 as described above, and inhibit PFA therapy 160 (e.g., stop the delivery of PFA energy as part of a PFA therapy routine and/or prevent initiating a current or further PFA therapy routine) based on comparing the sensor input 150 associated with the catheter distal end 134 to one or more threshold(s) associated with a position and/or movement of the catheter distal end 134. The sensor input 150 may be localization data that represents a position and/or orientation of the catheter distal end 134. According to such examples, the threshold(s) may correspond to one or more designated area of patient tissue. For example, the threshold(s) may correspond to one or more restricted areas of patient tissue surrounding one or more critical features such as electrical nodes, and the medical device system 100 is configured to inhibit PFA therapy if localization data indicates the catheter distal end 134 has entered the restricted area(s). According to other examples, the threshold(s) may correspond to one or more approved areas of patient tissue where PFA therapy is intended to be applied, and the medical device system 100 is configured to inhibit PFA therapy if localization data indicates that the catheter distal end 134 is positioned outside the approved areas. In still other examples, the threshold(s) may correspond to a distance of the catheter distal end from an initial position of a PFA therapy procedure, and the medical device system 100 is configured to inhibit PFA therapy comparing localization data to the distance.

In other examples, the threshold(s) may be associated with sensor input 150 including one or more measured forces on the catheter distal end 134 that indicate that the catheter distal end 134 has moved from an intended therapy position, e.g., that the catheter distal end 134 has slipped from a position in contact or close proximity to patient tissue where PFA therapy was intended to be delivered, which may indicate that the catheter distal end 134 might now be in contact with or close to other patient tissue that should not be ablated. As one example, the sensor input 150 may be associated with a contact force at a tip of the catheter distal end 134, and the threshold corresponds to a change amplitude and/or direction of the contact force. In other examples, the sensor input is associated with movement of the catheter distal end and the at least one threshold corresponds to movement in one or more of an X, Y, or Z direction.

In some examples, whether based on force or localization data, the threshold(s) may be selectable by an operator of the medical device system 100. For example, the system 100 may be configured to display an image representing the threshold(s) to an operator via a display, and enable the operator to increase or decrease the threshold(s). As one example, the system 100 may be configured to present an image with a proposed designated area (as a restricted area, approved area, and/or a distance D from an initial from a therapy routine) to an operator via a display, and receive user input increasing and/or decreasing the designated area relative to the proposed designated area.

The medical device system 100 depicted in FIG. 1 may deliver PFA therapy via the catheter 130 with increased safety in comparison to traditional techniques for PFA delivery. The medical device system 100 is uniquely configured to beneficially deliver PFA therapy while minimizing and/or eliminating a risk that PFA energy is inadvertently delivered to unintended patient tissue.

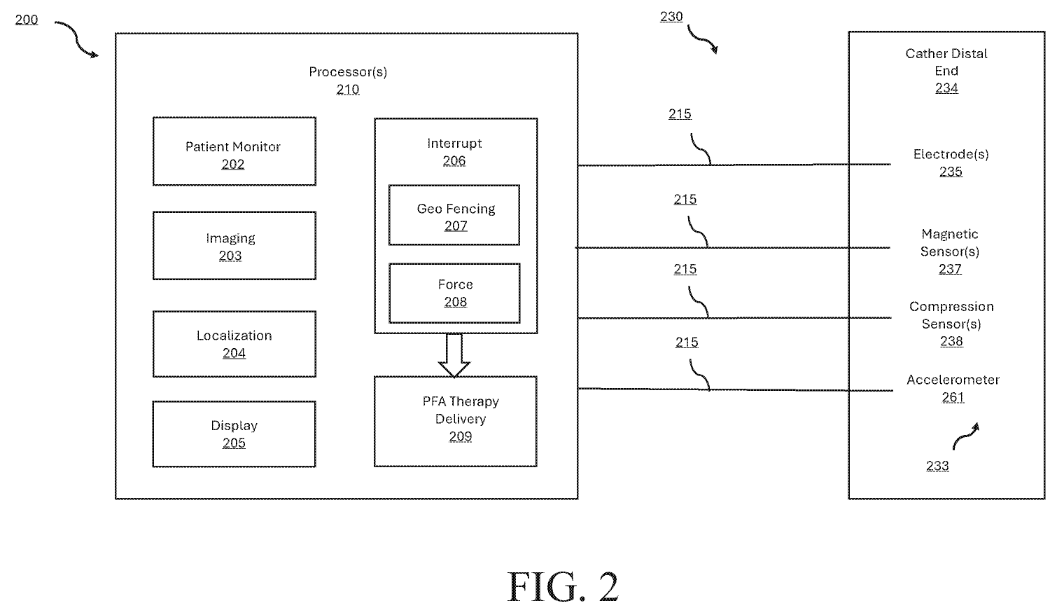

FIG. 2 is a block diagram depicting one example of a medical device system 200 configured to inhibit delivery of PFA therapy via a catheter 230 based on sensor input. As shown in FIG. 2, the medical device system 200 includes a catheter 230 with a catheter distal end 234 that corresponds to the catheter 130 depicted in FIG. 1. The catheter 130 may also correspond to other types of catheters not depicted herein that might be used to perform PFA ablation.

In the example of FIG. 2, the catheter distal end 234 includes a plurality of sensor(s) 233 which may be used to perform measurements, delivery therapy and/or other purposes as well. In the example of FIG. 2, the sensor(s) 233 may be coupled to one or more processor(s) 210 of the medical device system 200 via conductors 215 which may be housed within one or more lumen(s) that extend through a shaft 131 of the catheter 230. Although not depicted in FIG. 2, the medical device system 200 may further include analog and/or digital circuitry configured to condition analog signal data from the sensor(s) 233 for use by the processor(s) 210, including analog to digital conversion circuitry configured to digitize analog electrical signals for use by the processor(s) 210.

The processor(s) 210 may include one or more processing components configured to execute instructions stored in a memory component (not shown in FIG. 2) to perform the functions of the medical device system 200. For example, the processor(s) 210 may include one or more of a microcontroller, microprocessor, central processing unit (CPU), graphics processing unit (GPU), tencore processor (TPU), Field Programmable Gate Array (FPGA), Application Specific Integrated Circuit (ASIC) or the like configured to execute instructions stored in a memory to cause the medical device system 200 to perform the functions described. In some examples, the processor(s) 210 may be positioned local to the catheter 230, for example as part of the handle 140 shown in the FIG. 1 example and/or in local equipment coupled to the handle 140. In other examples, the processor(s) 210 may be positioned elsewhere and coupled to the medical device system 200 via a wired or wireless communications network.

The example of FIG. 2 depicts various modules 203-209 that represent functions of the medical device system 200 that may be executed by the processor(s) 210. As shown in FIG. 2, the medical device system 200 includes a patient monitor module 202, an imaging module 203, a localization module 204, a display module 205, an interrupt module 206, and a PFA therapy delivery module 209.

The patient monitor module 202 is configured to monitor sensor data from the sensor(s) 233 to provide information to one or more operator(s) of the medical device system 200 to assist the operator(s) to perform medical procedures. For example, the patient monitor module 202 may process input signals from the electrode(s) 235 (which may be ring electrodes 135 and/or tip electrodes 136) to detect an electrogram (EGM) signal of a patient, which represents electrical activity of the cardiac muscle and communicate the same to an operator. The patient monitor module 202 may process input signals (e.g., impedance signals) from the electrode(s) 235 and/or electrodes of a separate mapping catheter (not shown) to generate a map of a patient tissue. In some examples, the patient monitor module 202 may use such mapping data to identify candidate areas for ablation. The patient monitor module 202 may further measure, using the electrode(s) 235 or other sensors, other patient characteristics such as a heart rate, blood pressure, blood oxygen level, temperature, or other characteristic and communicate the same to operator(s) to assist the operator(s) to evaluate patient health as part of a medical procedure. For example, the display module 205 shown in FIG. 2 may be operable to control one or more display(s) (not shown) visible to operator(s) while performing a medical procedure, and control the display to output text, plots, or other image data to communicate sensed patient data or other information to an operator.

As shown in FIG. 2, the medical device system 200 further includes an imaging module 203. The imaging module 203 is configured to control one or more imaging systems (not shown) of the medical device system 200 to capture image data of a patient, including of internal organs, implantable medical devices (including catheter 230), or other structures within the body of a patient. For example, the imaging module 203 may be configured to receive image data from an ultrasound, magnetic resonance, X-Ray, fluoroscopy, or other type of non-invasive imaging system that enables image data representing internal structures to be captured. The imaging module 203 may be configured to process captured image data and cause the display module 205 to control one or more display(s) to present captured image data in real-time as an operator performs a medical procedure, for example, including when the catheter distal end 234 has been positioned within an organ or other bodily structure in a patient. In some examples, the imaging module 203 may generate images for presentation via the display(s) based on mapping data from the patient monitor module 202 as described above, for example by identifying in images one or more areas of patient tissue as a candidate for ablation therapy.

As shown in FIG. 2, the medical device system 200 further includes a localization module 204. The localization module 204 is configured to receive location data from the sensor(s) 233 to determine a position and/or orientation of the catheter distal end 234 in space. For example, the localization module 204 may receive localization data in the form of impedance data measured by the electrode(s) 235 responsive to one or more externally generated electric fields. For example, patch electrodes (not shown) may be secured at positions around the body of a patient to generate electric fields in substantially orthogonal X, Y, and Z dimensions, and the localization module 204 may detect impedance data from the electrode(s) 235 to determine a position/orientation of the catheter distal end relative to a three-dimensional reference grid defined by the substantially orthogonal electric fields.

In some examples, the localization module 204 may also, or instead, use signals from the magnetic sensor(s) 237 received via a conductor 215 to determine a position and/or orientation of the catheter distal end 234. For example, localization module 204 may receive location data from the magnetic sensor(s) 237 via conductor(s) 215 responsive to one or more externally generated magnetic fields. According to these examples, the medical device system 200 may include one or more components configured to generate magnetic fields in substantially orthogonal X, Y, and Z dimensions, and the localization module 204 uses location data from the magnetic sensor(s) 237 to determine a position/orientation of the catheter distal end 234 relative to a three-dimensional reference grid defined by the substantially orthogonal magnetic fields. In some examples, the medical device system 200 may be configured to use impedance/magnetic location data from both the electrode(s) 235 and the magnetic sensor(s) 237 relative to the respective electric and magnetic fields to a measure a position and/or orientation of the catheter distal end 234 with greater precision.

In some examples, position and/or orientation information from the localization module 204 may be used by the medical device system 200 to assist an operator to guide and/or position the catheter 230 within the body of a patient as part of a medical procedure. For example, the imaging module 203 may capture image data of internal organs and/or the catheter 230 as an operator inserts the catheter 230 into a vein or artery and advances the catheter distal end 234 to a desired position in contact or proximity to patient tissue including cardiac tissue in some examples. While the catheter 230 is being advanced or otherwise positioned, the imaging module 203 may receive position/orientation data from the localization module 204, and use the position data to generate images that accurately show a position of the catheter distal end 234 superimposed over images showing patient organs and/or tissue. The display module 205 may control one or more displays to present these images to an operator, who may use the images to orient/position the catheter 230 as part of a medical procedure. For example, the display module 205 may control the display(s) to present images showing the tip 139 of the catheter distal end 134 pressed against patient tissue as part of a medical procedure to deliver PFA therapy to the patient tissue.

As also shown in FIG. 2, the medical device system 200 includes a PFA therapy module 209. The PFA therapy module 209 is configured to cause PFA energy to be delivered to patient tissue via one or more electrode(s) 235. The PFA therapy module 209 may be configured to deliver energy in the form of pulses or relative high amplitude to ablate patient tissue, which may immediately damage or kill patient tissue cells when it is delivered to patient tissue. For example, the PFA therapy module 209 may be configured to treat arrhythmia conditions in a patient, which symptoms of which may include irregular heartbeats such as atrial fibrillation and/or atrial flutter.

A physician may diagnose cardiac arrhythmia by identifying inconsistencies in an electrocardiogram signal that indicate irregular heart activity, for example that is communicated to an operator by the patient monitor module 202 via a display or other user interface. To treat the arrhythmia, the physician may use the catheter 230, specifically signals measured through the catheter electrode(s) 235, to identify and ablate specific areas of cardiac tissue to enable natural electrical patterns to properly propagate through the tissue to eliminate or reduce the irregular activity. The PFA therapy module 209 may be configured to deliver a specific type of ablation energy to cardiac tissue in the form of relatively high amplitude pulses. In some examples, unlike other ablation techniques like RF or thermal ablation, the pulsed energy delivered by the PFA therapy module 209 may immediately damage and/or kill targeted cells. PFA therapy delivered to unintended patient tissue that contains critical neurological structures like a cardiac node may cause irreversible damage and require a pacemaker to be installed in the patient, potentially for life.

As shown in FIG. 2, the medical device system 200 further includes an interrupt module 206. The interrupt module 206 is configured to detect input from the sensor(s) 233 associated with the catheter distal end 234, and inhibit delivery of PFA energy based on comparing the sensor input to one or more threshold(s). If the sensor input indicates there may be a possibility that PFA energy might be delivered to unintended patient tissue, the interrupt module 206 inhibits the delivery of PFA energy by the catheter 230. For example, the interrupt module 206 may generate an interrupt signal that causes the PFA therapy module 209 to halt an already started therapy routine (e.g., stop the delivery any further pulse of PFA energy). In other examples, the interrupt signal may cause one or more therapy routines that have not yet been initiated from starting (e.g., to deliver PFA energy to patient tissue). In some examples, the interrupt module 206 may prevent any further PFA therapy routines until the sensor data indicates that the catheter distal end 134 has been repositioned or otherwise reset to deliver PFA energy to patient tissue intended for ablation.

Referring back to FIG. 2, the interrupt module 206 may include one or more of a geofencing module 207 and a force module 208. The geofencing module 207 may receive sensor data in the form of localization data from the sensor(s) 233 indicating a position/orientation of the catheter distal end 234, for example based on localization data from the electrode(s) 235 and/or magnetic sensor(s) 237 including for example coordinates relative to a three-dimensional reference grid defined by externally generated electric and/or magnetic fields. The geofencing module 207 may compare such localization data to one or more designated areas as a threshold, and inhibit the PFA therapy module 209 from delivering PFA energy based on whether the catheter distal end 234 is within or outside of the designated areas. For example, the designated areas may specify a restricted area of patient tissue (where PFA therapy should not be delivered) as a threshold and the geofencing module 207 inhibits the delivery of PFA therapy if the localization data indicates that the catheter distal end 234 has entered the restricted area. In other examples, the geofenced area may specify an approved area of patient tissue, and the geofencing module 207 inhibits the delivery of PFA therapy if the localization data indicates that the catheter distal end 234 is positioned outside of the approved area of patient tissue. In still other examples, the geofencing module 207 may compare localization data to both a restricted and approved areas as thresholds and inhibit the delivery of PFA energy based on comparing a position of the catheter distal end 234 to one or both, for example, by inhibiting the delivery of PFA energy if catheter distal end 234 exits an approved area and/or enters a restricted area.

The force module 208 is configured to receive sensor data in the form of force data from one or more sensor(s) 233 indicating movement of the catheter distal end 234, for example based on force data associated with the catheter distal end 234. For example, the force data may be from a contact force sensor 238 configured to measure a magnitude and direction of a force on the tip 139 (referring to FIG. 1, not represented in FIG. 2) of the catheter distal end 234, for example when the tip 139 is pressed against patient tissue. For example, the contact force sensor 238 may generate electrical signals representing at least three measurable fiber optic gaps that indicate each indicate a magnitude of contact force and enable a direction of the contact force to be determined. In other examples, the force sensor may also or instead be an accelerometer 261 configured to indicate force(s) associated movement and direction of movement along x, y, and z axes. The force module 208 may compare such force data to one or more force thresholds indicative of sudden movement/slipping of the catheter distal end 234, and inhibit the PFA therapy module 209 from delivering PFA therapy based on the comparison. For example, the one or more thresholds may be associated with measured force signals that indicate that the catheter distal end 234 has changed position quickly, (e.g., slipped) from a position in contact with patient tissue intended for ablation.

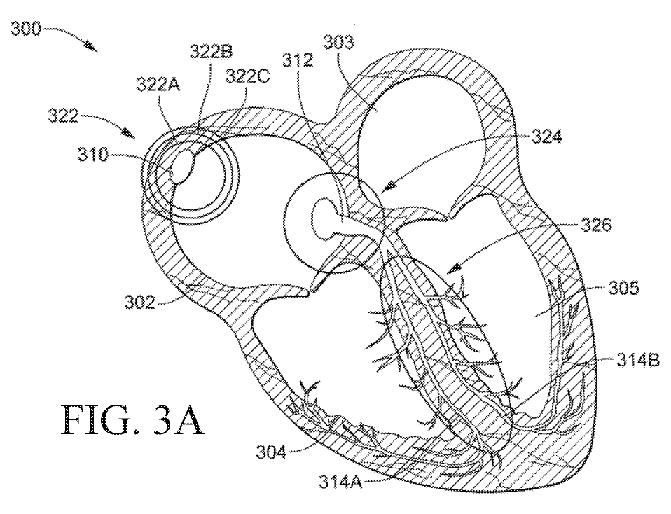

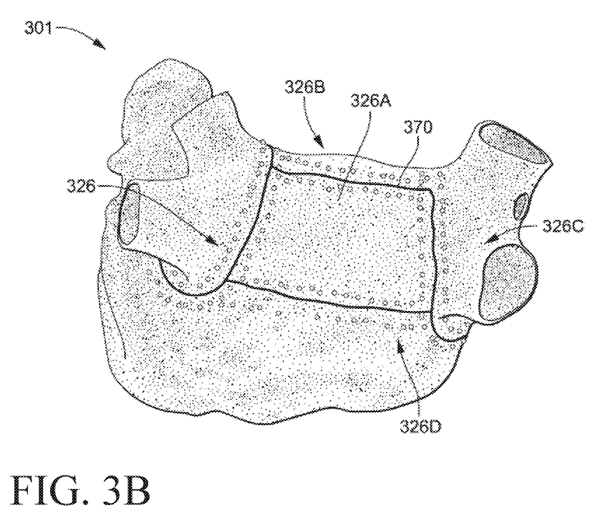

FIG. 3A is a diagram showing one example a 2D cross-sectional image 300 of a human cardiac muscle and a restricted area 322. The image 300 may be generated by the imaging module the imaging module 203 depicted in FIG. 2, and presented to one or more operator(s) via display controlled by the display module 205. The image 300 may be generated based on captured image data of internal organs or other structures in a patient. The example of FIG. 3A is presented as a 2D image for simplicity. In other examples, the display module 205 may present the image 300 as a 3D cross section or other view of a cardiac muscle.

As shown in FIG. 3A, the depicted cardiac muscle includes a right atrium 302, a left atrium 303, a right ventricle 304, and a left ventricle 305. As shown in FIG. 3A, the right atrium 302 includes two nodes 310 and 312. The node 310 is a sino atrial (SA) node that may act as a primary pacemaker of the heart that generates electrical impulses that stimulate the atria 302, 303 to contract. The node 312 is an atrioventricular (AV) node situated at a junction between the atria 302, 303, and the ventricles 304, 305 and functions to receive electrical signals from the SA node 310 and delay their transmission to the ventricles 304, 305. As shown in FIG. 3A, the depicted cardiac muscle further includes a bundle of His nerves 314A, 314B that extend from the AV node 312 into the ventricles 304, 305, and may further distribute electrical impulses to ventricular muscle.

In the example of FIG. 3A, system 200 (e.g., geofencing module 207) has identified a plurality of geofenced areas of patient tissue as restricted areas 322, 324 and/or 326 which operate as a threshold for the medical device system 200 to inhibit the delivery of PFA therapy based on comparison to sensor data that indicates a position and/or orientation of the catheter distal end 234. For example, the system 200 may inhibit PFA therapy if the sensor data indicates that the catheter distal end 234 has entered the restricted areas 322, 324 and/or 326 to prevent cardiac tissue within the restricted zones from being ablated. As shown in FIG. 3A, the system 200 has arranged the restricted areas 322, 324 and/or 326 to surround critical features of the cardiac muscle, including one or more of the SA node 310, the VA node 312, and the His nerves 314A, 314B to prevent accidental damage to the critical features.

In some examples, the system 200 may be configured to automatically identify critical structures in a patient’s cardiac muscle such as the nodes 310, 312 and/or His nerves 314A, 314B and automatically determine the restricted area(s) 322, 324 and/or 326 to inhibit delivery of PFA therapy should sensor data indicate that the catheter distal end 234 has entered the restricted area(s) 322, 324 and/or 326. In other examples, as shown by the restricted area 322 surrounding the SA node 310, in FIG. 3A, a size and/or relative position of the restricted area(s) 322, 324, and/or 326 may be selectable based on operator input. For example, as shown in FIG. 3A, the system 200 may present an image showing an initial proposal for a restricted area 322B to surround the SA node 310, and enable a user to increase or decrease the proposed restricted area to make it larger as shown by restricted area 322C, or smaller as shown by the restricted area 322A, or otherwise select a desired size, shape, or other characteristic of the restricted area 322. In some examples, each of the restricted area(s) 322, 324 and/or 326 depicted in FIG. 3A may be selectable by a user to increase, decrease, or otherwise specify a size, location, diameter, volume (when 3D), or other characteristic of the restricted area(s) 322, 324 and/or 326.

FIG. 3A shows one example where the image 301 shows a cardiac muscle as a 2D cross section and the restricted area with the shape of a circle. In other examples, one or more of the restricted area(s) 322, 324 and/or 326 may be represented by another shape relative to the two-dimensional cross section of the image 301, for example a square, rectangle, or other 2D shape. In other examples, the image 301 may be shown as a 3D cross-section or otherwise, and the restricted areas 322, 324 and/or 326 may be shown as a 3D sphere, cube, cylinder, or other 3D shape relative to the 3D image. Such a 3D representation of a restricted area 322, 324 and/or 326 may similarly be made selectable by a user, for example to change the volumetric size, shape, position, or other characteristic of a 3D representation of the restricted areas 322, 324 and/or 326.

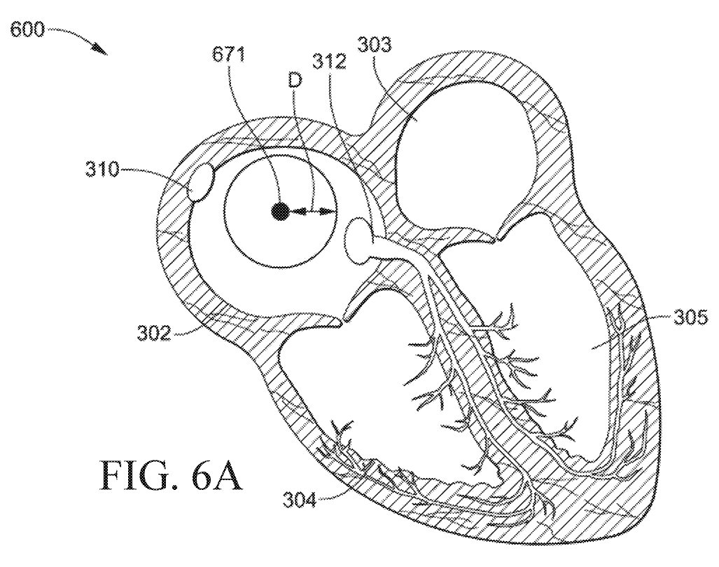

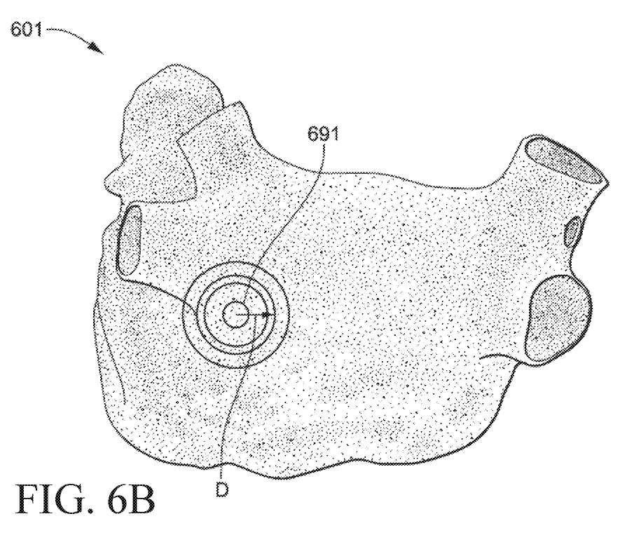

FIG. 3B is a diagram that depicts one example of a 3D image 301 of a posterior view of the left atrium (303, as shown in FIG. 3A) looking at the pulmonary veins of a cardiac muscle and a restricted area 362A of patient tissue. The example of FIG. 6 represents a zoomed in view into the left atrium 303. As shown in FIG. 3B, the restricted area 362A is bounded by dotted lines on all sides. The restricted area 362A has a substantially rectangular shape, which may instead be substantially cubic, cylindrical and/or another shape if represented in 3D. Also shown in FIG. 3B is a path 370 showing where an operator/physician may intend to deliver PFA therapy to treat a patient. As shown in FIG. 3A, the path 370 remains outside of the restricted area 362A. If, during a PFA therapy procedure, localization data indicates that a position of the catheter distal end 234 has entered the restricted area 362A, system 200 may inhibit the PFA therapy to avoid a possibility that PFA energy is inadvertently delivered to unintended patient tissue, potentially causing irreversible damage.

As shown in FIG. 3B, the system 200 may be configured to determine additional restricted areas beyond the restricted area 362A. For example, the system 200 may determine additional restricted areas surrounding a SA node, VA node, bundle of His fibers, or any other cardiac structure or tissue as shown in FIG. 3A. In some examples, the system may determine additional restricted areas to bound an intended ablation path 370 on all sides. The ablation path 370 shown in FIG. 3B may continue to the undepicted side of the heart muscle in some examples. In some examples, as shown in FIG. 3B, the system 200 may determine further restricted areas 362B, 362C, 362D, and 362E that along with the restricted area 362A surround the intended ablation path 370 on all sides such that unintended movement of the catheter distal end 234 away from the intended ablation path 370 in any direction causes delivery of PFA therapy to be inhibited.

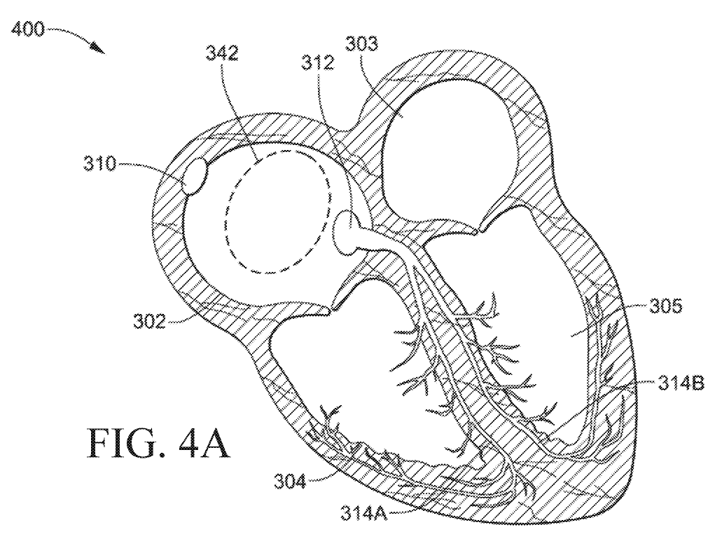

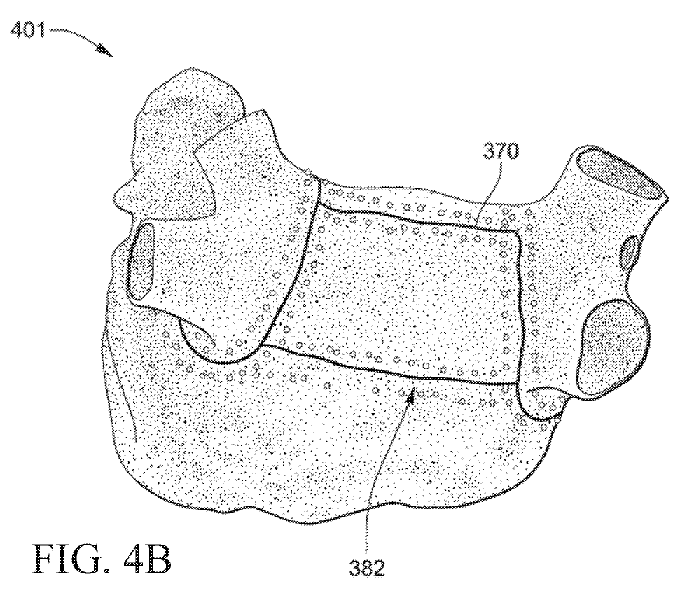

FIG. 4A is a diagram showing one example of a 2D cross-section image 400 of a human cardiac muscle and an approved area 342. The image 400 is substantially similar to image 300 depicted in FIG. 3A and may similarly be generated by the imaging module 203 depicted in FIG. 2, and presented to one or more operator(s) via display controlled by the display module 205. The image 400 may be generated based on captured image data of internal organs or other structures in a patient. Like FIG. 3A, FIG. 4A is presented as a 2D image for simplicity. In other examples, the display module 205 may present the image 400 as a 3D cross section or other 3D view.

The example of FIG. 4A differs from the example of FIG. 3A in that system 200 is configured to inhibit delivery of PFA therapy based on comparing sensor data to an approved therapy area 342 as a threshold for comparison to sensor data. According to these examples, the geofencing module 207 may trigger an interrupt signal to inhibit delivery of PFA therapy if localization sensor data indicates that the catheter distal end 234 is no longer in the approved therapy area 342, i.e., the catheter distal end 234 has exited the approved therapy area 342. As shown in the example of FIG. 4A, the approved therapy area 342 defines patient tissue positioned in the right atrium 302 distanced from SA node 310 and the VA node 312 where PFA therapy is unlikely to impact the nodes 310, 312 and cause potentially irreversible damage. In other examples not depicted, system 200 may enable additional approved therapy areas corresponding to different patient tissue positioned to avoid critical cardiac structures such as the nodes 310, 312, bundle of His nerves 314A, 314B, or other critical cardiac structures.

FIG. 4B is a diagram that depicts one example of a 3D image 401 of a posterior view of the left atrium (303), as shown in FIG. 4A) looking at the pulmonary veins of a of a cardiac muscle and an approved area 382 of patient tissue. As shown in FIG. 4B, the approved therapy area 382 is bounded by the dotted lines on all sides. The approved therapy area 382 has a substantially rectangular shape in FIG. 4B but may be other shapes in other examples. In some examples, the approved therapy area 382 may instead be circular, cubic, cylindrical or another shape in 3D examples. In the example of FIG. 4B, the approved therapy area 382 defines a path 370 showing where an operator/physician may deliver PFA therapy to treat a patient. As shown in FIG. 4B, the path 370 traverses patient tissue inside of the approved therapy area 382. If, during a PFA therapy procedure, localization data indicates that the catheter distal end 234 is positioned outside of the approved therapy area 382, system 200 may inhibit the PFA therapy.

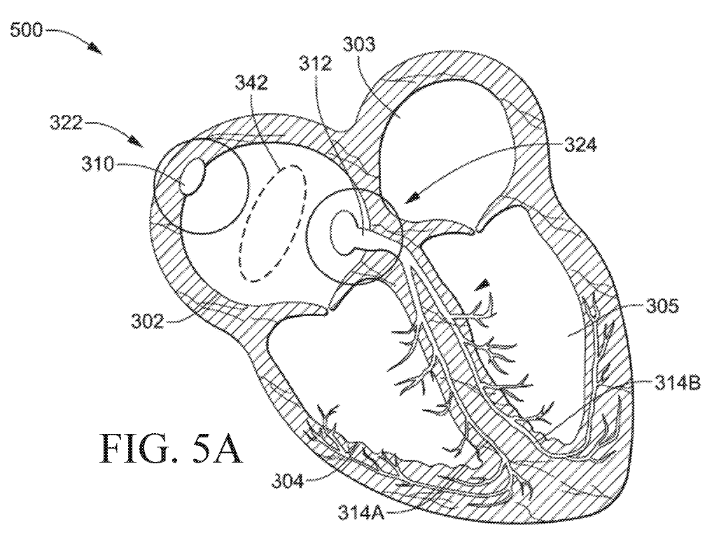

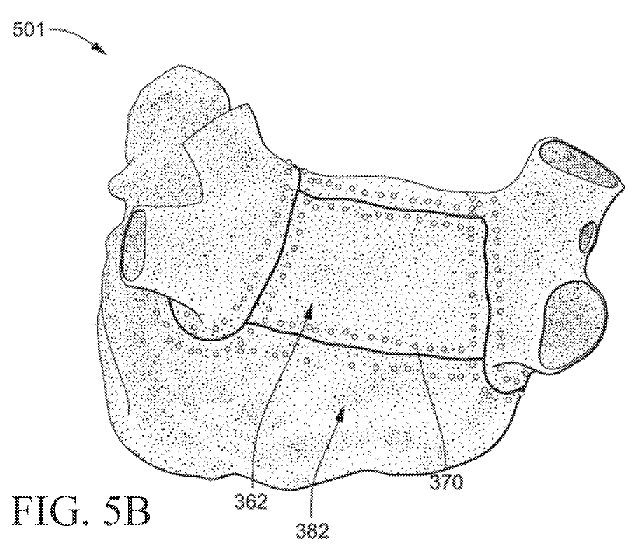

FIG. 5A is a diagram showing one example a 2D cross-sectional image 500 of a human cardiac muscle, a restricted area 322 of patient tissue and an approved therapy area 342 of patient tissue. The image 500 is substantially similar to the images 300, 400 depicted in FIGS. 3A and 4A and may similarly be generated by the imaging module 203 depicted in FIG. 2 and presented to one or more operator(s) via a display controlled by the display module 205. The image 400 may be generated based on captured image data of internal organs or other structures in a patient. Like FIGS. 3A and 4A, FIG. 5A is presented as a 2D image for simplicity purposes. In other examples, the display module 205 may present the image 500 as a 3D cross section or other 3D view.

The example of FIG. 5A differs from the example of FIGS. 3A and 4A in that system 200 is configured to inhibit delivery of PFA therapy based on comparing sensor data to both a restricted area 322 and an approved therapy area 342 as thresholds for comparison to sensor data. According to these examples, the geofencing module 207 may trigger an interrupt signal to inhibit delivery of PFA therapy if localization sensor data indicates that the catheter distal end 234 has entered a restricted area 322 and/or is no longer in an approved therapy area 342. As shown in the example of FIG. 5A, the restricted therapy area 342 defines patient tissue positioned in the right atrium distanced from SA node 310 and the VA node 312 where PFA therapy is unlikely to impact the nodes 310, 312 and cause potentially irreversible damage. As also shown in FIG. 5A, the approved therapy area 342 is arranged between the restricted areas 322 and 324 which surround the SA node 310 and the AV node, respectively. In other examples not depicted, system 200 may enable one or more additional restricted areas 322 and/or approved therapy areas 342 corresponding to different patient tissue positioned to avoid critical cardiac structures such as the nodes 310, 312, bundle of His nerves 314A, 314B, or other critical cardiac structures or tissue.

FIG. 5B is a diagram that depicts one example of a three-dimensional image 501 of a posterior view of the left atrium (303), as shown in FIG. 4A) looking at the pulmonary veins of a of a cardiac muscle, a restricted area 362 and an approved area 382 of patient tissue. As shown in FIG. 5B, the approved therapy area 382 is bounded by the dotted lines on all sides. In the example of FIG. 5B, the approved therapy area 382 and the restricted area 362 define a path 370 showing where an operator/physician may intend to deliver PFA therapy to treat a patient. As shown in FIG. 5B, the path 370 remains inside of the approved therapy area 382 and outside of the restricted area 362. If, during a PFA therapy procedure, localization data indicates that a position of the catheter distal end 234 has entered the restricted area 362 and/or exited the approved area 382, system 200 may inhibit the PFA therapy to avoid a possibility that PFA therapy is inadvertently delivered to the nodes 310, 312, bundle of His nerves 314A, 314B or other critical cardiac structures or tissue.

FIG. 6A is a diagram showing one example a 2D cross-section image 600 of a human cardiac muscle, an initial position 671 of a PFA therapy procedure, and a distance D of the catheter distal end from the initial position 671. The image 600 is substantially similar to images 300, 400, and 500 depicted in FIGS. 3A, 4A, and 5A and may similarly be generated by the imaging module 203 depicted in FIG. 2, and presented to one or more operator(s) via display controlled by the display module 205. The image 600 may be generated based on captured image data of internal organs or other structures in a patient. Like FIGS. 3A, 4A, and 5A, FIG. 6A is presented as a 2D image for simplicity. In other examples, the display module 205 may present the image 600 as a 3D cross section or other 3D view.

The example of FIG. 6A differs from the example of FIGS. 3A, 4A, and 5A in that system 200 is configured to inhibit delivery of PFA energy based on comparing sensor data to a distance D from an initial position 671 of a PFA therapy procedure as a threshold for comparison to sensor data. According to these examples, the geofencing module 207 may trigger an interrupt signal to inhibit delivery of PFA therapy if localization sensor data indicates that the catheter distal end 234 has moved the distance D or more than the distance D from the initial position 671. As shown in the example of FIG. 6A, the distance D may be selected to define an area of patient tissue surrounding the initial position 671 positioned in the right atrium 302 distanced from SA node 310 and the VA node 312 where PFA therapy is unlikely to impact the nodes 310, 312 and cause potentially irreversible damage. In other examples not depicted, system 200 may position the initial position 671 and select the distance D to correspond to other areas of patient tissue positioned to avoid delivery of PFA therapy to critical cardiac structures such as the nodes 310, 312, bundle of His nerves 314A, 314B, or other critical cardiac structures. In some examples, the initial position 671 and/or distance D may be selectable by an operator. For example, the system 200 may be configured to cause the image 600 to be projected on a display, and enable the user to increase or decrease the distance D based on user input as needed.

FIG. 6B is a diagram that depicts one example of a three-dimensional image 601 of a posterior view of the left atrium (303, as shown in FIG. 6A) looking at the pulmonary veins of a of a cardiac muscle 3D and an initial position 691 for the delivery of a PFA therapy routine. As shown in FIG. 6B, multiple distances D are shown from the initial position 691. In some examples, the image 601 may be presented to an operator of the medical device system 200 with one or more proposals for a distance D, which may be changed/selected by the operator. As shown in FIG. 6B the distance D may be selected to avoid delivery of PFA energy unintended tissue. Once the distance D is determined based on user input or otherwise, the geofencing module 207 may trigger an interrupt signal to inhibit delivery of PFA therapy if localization sensor data indicates that the catheter distal end 234 has moved more than the distance D from the initial position 671.

Referring back to FIG. 2, the interrupt module 206 may include a force module 208 configured to receive sensor data in the form of data from one or more sensor(s) 233 indicating movement of the catheter distal end 234, for example based on force data associated with the catheter distal end 234.

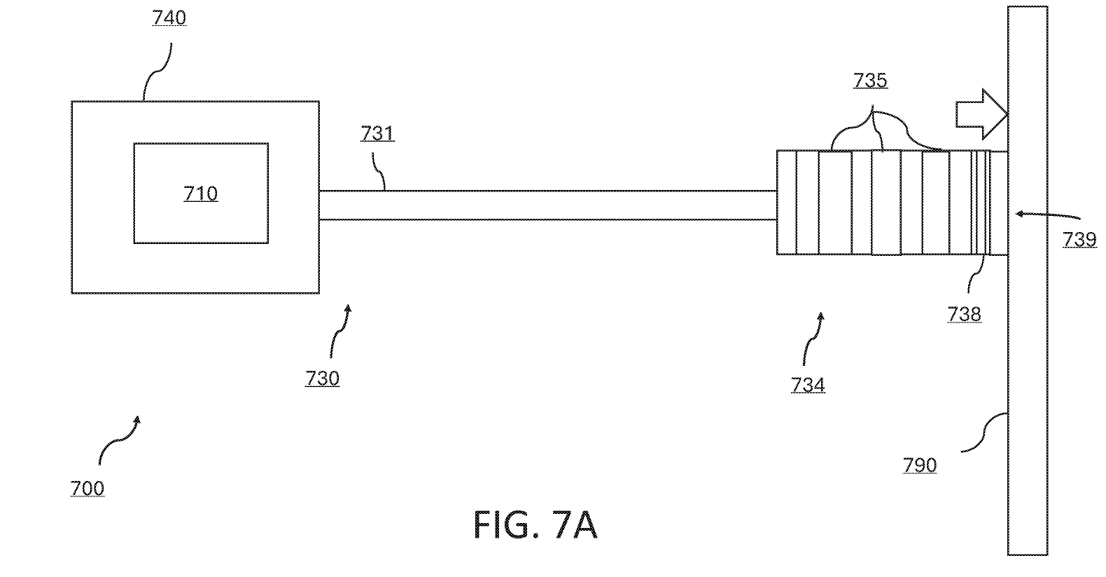

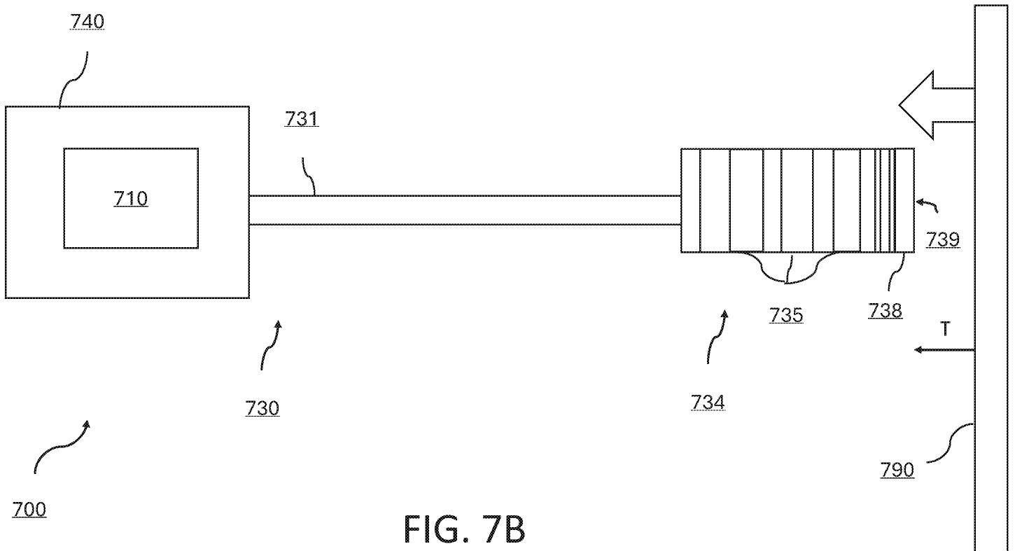

FIGS. 7A-7B depict one example of medical device system 700 with a catheter 730 with a catheter distal end 734 that includes a contact force sensor 738 to measure force. As shown in the example of FIG. 7A, the catheter 730 corresponds to the catheter depicted in FIG. 1 and includes a handle 740, one or more processor(s) 710, a shaft 731, and a catheter distal end 734. The catheter distal end 734 includes sensors like ring electrodes 735 shown in FIGS. 7A-7B but may also include electrodes in the tip 739 of the catheter distal end 734, magnetic sensor(s) such as one or more magnetic coil(s), or other sensors configured to capture data. The contact force sensor 738 is configured to detect a magnitude and/or direction of force upon the tip 739. As one example, the contact force sensor 738 may include at least three optical gaps configured to be measured to determine a direction and/or magnitude of contact force on the tip 739 for example in response to an operator/physician pressing the catheter distal end 734 against patient tissue 790. In some examples, a direction from which a contact force is applied to the tip 739 may indicate an angle of the catheter distal end 734 relative to the patient tissue and be determined by differences in force measured by the respective optical fiber gaps.

In the example of FIG. 7A, the tip 739 is compressed against patient tissue such that the contact force sensor 738 measures a force against the tip 739. As shown in FIG. 7B, the catheter distal end 734 has moved such that the contact force sensor 738 may detect little or no force upon the tip 739. Referring back to FIG. 2, the force module 208 may monitor force data from the contact force sensor 738 to compare the force data to one or more thresholds to trigger an interrupt signal that inhibits delivery of PFA therapy. For example, if a magnitude and/or direction of force measured by the contact force sensor 738 falls below a threshold that indicates the catheter distal end 734 is no longer in contact and/or in poor contact with patient tissue 790, the force module 208 may cause PFA therapy to be inhibited.

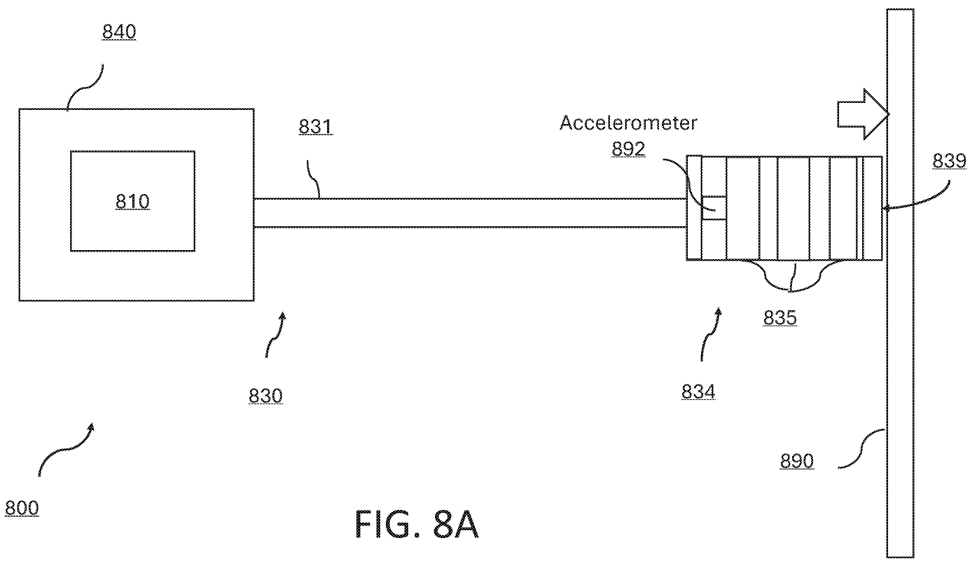

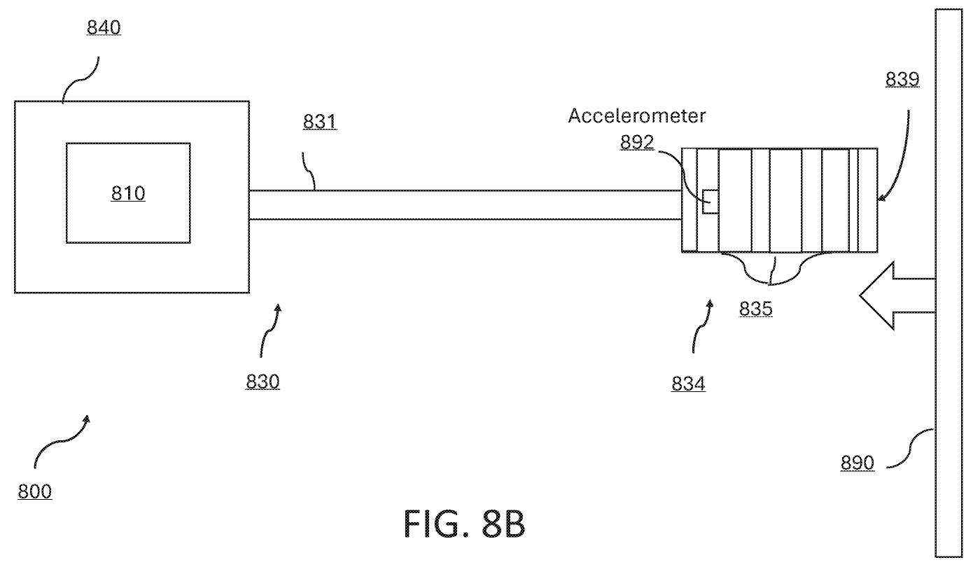

FIGS. 8A and 8B depict one example of a medical device system 800 with a catheter 830 with a catheter distal end 834 that includes an accelerometer sensor 892 used as a force sensor to detect forces associated with movement of the catheter distal end 834. As shown in the example of FIG. 8A, the catheter 830 corresponds to the catheter 130 depicted in FIG. 1 and includes a handle 840, one or more processors 810, a shaft 831, and a catheter distal end 834. The catheter distal end 834 includes sensors like ring electrodes 835 shown in FIGS. 8A-8B but may include electrodes in the tip 839 of the catheter distal end 834, magnetic sensor(s) such as one or more magnetic coil(s), or other sensors configured to capture data. The accelerometer sensor 892 is configured to detect a magnitude and/or direction of force along three orthogonal axes (e.g., X, Y, and Z axes).

In the example of FIG. 8A, the tip 839 is compressed against patient tissue 890 such that the accelerometer sensor 892 does not measure any forces due to movement of the catheter distal end 834 relative to the patient tissue 890. As shown in FIG. 8B, the catheter distal end 834 has moved away from the patient tissue 890 such that and the accelerometer sensor 892 senses forces associated with movement of the catheter distal end 834. According to such examples, the force module 208 may be configured to compare force data from the accelerometer sensor 892 to one or more thresholds associated with movement of the catheter distal end 834 to a position that might risk of that unintended tissue is mistakenly ablated, and accordingly cause an interrupt signal to be sent to the PFA therapy module 209 to inhibit the delivery of PFA energy.

Referring back to FIG. 2, the medical device system 200 may be configured to use the various techniques described herein for preventing PFA energy from being delivered to unintended tissue may be used alone or in combination. For example, the medical device system 200 may be configured to present an operator with options for how the operator wants to configure the system. For example, the medical device system 200 may provide a user with an option to use geotagging to define a restricted area, approved therapy area, or both to inhibit delivery of PFA energy. The medical device system 200 may further provide the user to, also or instead, use measured forces from one or more force sensors to inhibit the delivery of PFA therapy. Regardless of whether force data, location data, or other data are used to inhibit the delivery of PFA energy, thresholds associated with inhibiting the delivery of PFA therapy may be selectable by a user. For example, thresholds corresponding to an area, volume and/location designated area of patient tissue may be selected by a user. As another example, an amount and/or direction of movement to inhibit the delivery of PFA therapy may also be selectable by a user.

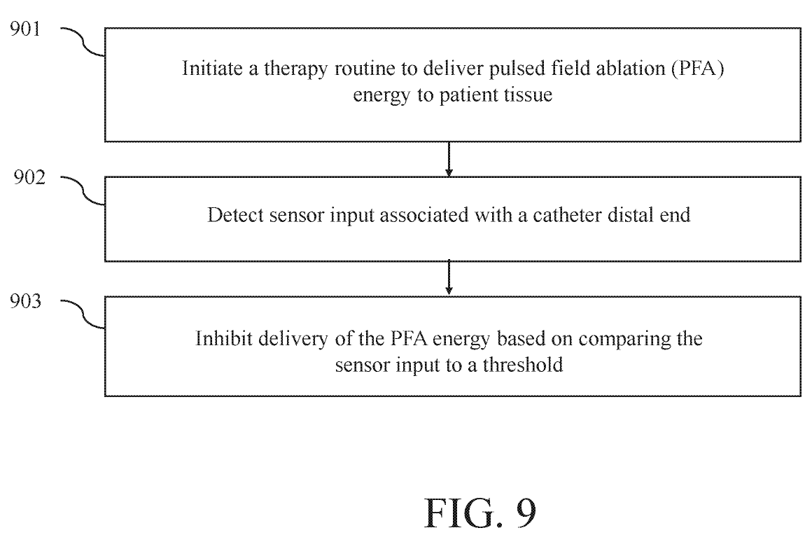

FIG. 9 is a flow diagram that depicts one example of a method of operating a medical device system including a catheter 130 configured to deliver PFA energy. As shown in FIG. 9, at 901, the method includes initiating a therapy routine to deliver pulsed field ablation (PFA) energy to patient tissue. As also shown in FIG. 9, at 902, the method further includes detecting sensor input associated with the catheter distal end 234. As also shown in FIG. 9, at 903, the method further includes inhibiting delivery of the pulsed field ablation (PFA) energy based on comparing the sensor input to a threshold. In some examples, the sensor input indicates one or more of a position and orientation of the catheter distal end 234. In some examples, the method further includes comparing the sensor input to a threshold that corresponds to a designated area of patient tissue. In some examples, the method further includes receiving input from an operator to select the designated area of patient tissue. In some examples, the designated area is a restricted area 322 of patient tissue, and the method further includes inhibiting further delivery of the PFA energy responsive to the catheter distal end 234 entering the restricted area 322 of patient tissue. In some examples, the restricted area 322 surrounds at least one node 310, 312 of a heart muscle. In some examples, the threshold corresponds to an approved therapy area 342 of patient tissue, and the method further includes inhibiting further delivery of the PFA energy responsive to the catheter distal end 234 exiting the approved therapy area 342. In some examples, the method further includes presenting an image with a proposed designated area of a patient tissue to an operator via a display, and receiving user input increasing and/or decreasing the designated area relative to the proposed designated area. In some examples, the method further includes comparing the sensor input to a threshold that corresponds to a distance D between the catheter distal end 234 and an initial position 671 of the therapy routine. In some examples, method further includes comparing the sensor input to a threshold that corresponds to one or more measured forces associated with the catheter distal end 234. In some examples, method further includes comparing a contact force at a tip of the catheter distal end 234 to at least one threshold that corresponds to a change in one or more of amplitude and direction of the contact force. In some examples, method further includes comparing sensor input associated with movement of the catheter distal end in one or more of an X, Y, or Z directions to the at least one threshold.

The following relates to numbered clauses of the disclosure.

Clause 1. A medical device, comprising: a catheter distal end configured to be positioned in contact with or proximal to patient tissue within a heart muscle of a patient; and a processor configured to: initiate a therapy routine to deliver pulsed field ablation (PFA) energy to the patient tissue; receive sensor input associated with the catheter distal end, wherein the sensor input comprises a position of the catheter distal end; compare the position of the catheter distal end to a restricted area of patient tissue surrounding at least one cardiac conduction structure of the heart muscle; and inhibit delivery of the pulsed field ablation (PFA) energy responsive to the catheter distal end entering the restricted area of patient tissue surrounding the at least one cardiac conduction structure.

Clause 2. The medical device of clause 1, wherein the at least one cardiac conduction structure comprises at least one of a sinoatrial (SA) node, an atrioventricular (AV) node, or a bundle of His.

Clause 3. The medical device of any one of clauses 1 to 2, wherein the restricted area of patient tissue is selectable by an operator.

Clause 4. The medical device of clause 3, wherein the processor is further configured to: present an image with a proposed restricted area surrounding the at least one cardiac conduction structure to an operator via a display; and receive user input increasing or decreasing the restricted area relative to the proposed restricted area.

Clause 5. The medical device of any one of clauses 1 to 4, wherein the processor is further configured to: compare the position of the catheter distal end to an approved therapy area of patient tissue; and inhibit further delivery of the PFA energy responsive to the catheter distal end exiting the approved therapy area.

Clause 6. The medical device of clause 5, wherein the approved therapy area is positioned between a plurality of restricted areas, each restricted area surrounding a respective cardiac conduction structure.

Clause 7. The medical device of any one of clauses 1 to 6, wherein the sensor input further comprises an orientation of the catheter distal end.

Clause 8. The medical device of any one of clauses 1 to 7, wherein the catheter distal end comprises at least one of an electrode or a magnetic sensor configured to generate localization data representing the position of the catheter distal end.

Clause 9. A medical device, comprising: a catheter distal end configured to be positioned in contact with patient tissue; and a processor configured to: initiate a therapy routine to deliver pulsed field ablation (PFA) energy to the patient tissue; receive sensor input associated with the catheter distal end, wherein the sensor input comprises one or more measured forces associated with the catheter distal end; and inhibit delivery of the pulsed field ablation (PFA) energy based on comparing the one or more measured forces to a threshold indicative of movement of the catheter distal end from an intended therapy position.

Clause 10. The medical device of clause 9, wherein the sensor input is associated with a contact force at a tip of the catheter distal end, and the threshold corresponds to a change in one or more of amplitude and direction of the contact force.

Clause 11. The medical device of clause 10, wherein the catheter distal end comprises a contact force sensor configured to measure a magnitude and direction of force upon the tip.

Clause 12. The medical device of clause 11, wherein the contact force sensor comprises at least three fiber optic gaps configured to be measured to determine the magnitude and direction of the contact force.

Clause 13. The medical device of any one of clauses 9 to 12, wherein the sensor input is associated with movement of the catheter distal end, and the threshold corresponds to a movement of the catheter distal end in one or more of an X, Y, or Z direction.

Clause 14. The medical device of clause 13, wherein the catheter distal end comprises an accelerometer configured to detect forces associated with movement of the catheter distal end along three orthogonal axes.

Clause 15. The medical device of any one of clauses 9 to 14, wherein the threshold is selectable by an operator.

Clause 16. A method, comprising: initiating a therapy routine to deliver pulsed field ablation (PFA) energy to patient tissue within a heart muscle of a patient; receiving sensor input associated with a catheter distal end positioned within the heart muscle, wherein the sensor input comprises a position of the catheter distal end; comparing the position of the catheter distal end to a designated area of patient tissue associated with at least one cardiac conduction structure of the heart muscle; and inhibiting delivery of the pulsed field ablation (PFA) energy based on the comparing.

Clause 17. The method of clause 16, wherein the designated area is a restricted area of patient tissue surrounding the at least one cardiac conduction structure, and inhibiting delivery of the PFA energy comprises inhibiting further delivery of the PFA energy responsive to the catheter distal end entering the restricted area of patient tissue.

Clause 18. The method of clause 17, wherein the at least one cardiac conduction structure comprises at least one of a sinoatrial (SA) node, an atrioventricular (AV) node, or a bundle of His.

Clause 19. The method of any one of clauses 16 to 18, wherein the designated area is an approved therapy area of patient tissue, and inhibiting delivery of the PFA energy comprises inhibiting further delivery of the PFA energy responsive to the catheter distal end exiting the approved therapy area.

Clause 20. The method of any one of clauses 16 to 19, further comprising: presenting an image with a proposed designated area of the heart muscle to an operator via a display; and receiving user input increasing or decreasing the designated area relative to the proposed designated area.

While this invention has been described with reference to illustrative examples, this description is not intended to be construed in a limiting sense. Various modifications and combinations will be apparent to persons skilled in the art upon reference to the description. It is therefore intended that the appended claims encompass any such modifications.

1. A medical device, comprising:
a catheter distal end configured to be positioned in contact with or proximal to patient tissue within a heart muscle of a patient; and
a processor configured to:
initiate a therapy routine to deliver pulsed field ablation (PFA) energy to the patient tissue;
receive sensor input associated with the catheter distal end, wherein the sensor input comprises a position of the catheter distal end;
compare the position of the catheter distal end to a restricted area of patient tissue surrounding at least one cardiac conduction structure of the heart muscle; and
inhibit delivery of the pulsed field ablation (PFA) energy responsive to the catheter distal end entering the restricted area of patient tissue surrounding the at least one cardiac conduction structure.

2. The medical device of claim 1, wherein the at least one cardiac conduction structure comprises at least one of a sinoatrial (SA) node, an atrioventricular (AV) node, or a bundle of His.

3. The medical device of claim 1, wherein the restricted area of patient tissue is selectable by an operator.

4. The medical device of claim 3, wherein the processor is further configured to:
present an image with a proposed restricted area surrounding the at least one cardiac conduction structure to an operator via a display; and
receive user input increasing or decreasing the restricted area relative to the proposed restricted area.

5. The medical device of claim 1, wherein the processor is further configured to:
compare the position of the catheter distal end to an approved therapy area of patient tissue; and
inhibit further delivery of the PFA energy responsive to the catheter distal end exiting the approved therapy area.

6. The medical device of claim 5, wherein the approved therapy area is positioned between a plurality of restricted areas, each restricted area surrounding a respective cardiac conduction structure.

7. The medical device of claim 1, wherein the sensor input further comprises an orientation of the catheter distal end.

8. The medical device of claim 1, wherein the catheter distal end comprises at least one of an electrode or a magnetic sensor configured to generate localization data representing the position of the catheter distal end.

9. A medical device, comprising:
a catheter distal end configured to be positioned in contact with patient tissue; and
a processor configured to:
initiate a therapy routine to deliver pulsed field ablation (PFA) energy to the patient tissue;
receive sensor input associated with the catheter distal end, wherein the sensor input comprises one or more measured forces associated with the catheter distal end; and
inhibit delivery of the pulsed field ablation (PFA) energy based on comparing the one or more measured forces to a threshold indicative of movement of the catheter distal end from an intended therapy position.

10. The medical device of claim 9, wherein the sensor input is associated with a contact force at a tip of the catheter distal end, and the threshold corresponds to a change in one or more of amplitude and direction of the contact force.

11. The medical device of claim 10, wherein the catheter distal end comprises a contact force sensor configured to measure a magnitude and direction of force upon the tip.

12. The medical device of claim 11, wherein the contact force sensor comprises at least three fiber optic gaps configured to be measured to determine the magnitude and direction of the contact force.

13. The medical device of claim 9, wherein the sensor input is associated with movement of the catheter distal end, and the threshold corresponds to a movement of the catheter distal end in one or more of an X, Y, or Z direction.

14. The medical device of claim 13, wherein the catheter distal end comprises an accelerometer configured to detect forces associated with movement of the catheter distal end along three orthogonal axes.

15. The medical device of claim 9, wherein the threshold is selectable by an operator.

16. A method, comprising:
initiating a therapy routine to deliver pulsed field ablation (PFA) energy to patient tissue within a heart muscle of a patient;
receiving sensor input associated with a catheter distal end positioned within the heart muscle, wherein the sensor input comprises a position of the catheter distal end;
comparing the position of the catheter distal end to a designated area of patient tissue associated with at least one cardiac conduction structure of the heart muscle; and
inhibiting delivery of the pulsed field ablation (PFA) energy based on the comparing.

17. The method of claim 16, wherein the designated area is a restricted area of patient tissue surrounding the at least one cardiac conduction structure, and inhibiting delivery of the PFA energy comprises inhibiting further delivery of the PFA energy responsive to the catheter distal end entering the restricted area of patient tissue.

18. The method of claim 17, wherein the at least one cardiac conduction structure comprises at least one of a sinoatrial (SA) node, an atrioventricular (AV) node, or a bundle of His.

19. The method of claim 16, wherein the designated area is an approved therapy area of patient tissue, and inhibiting delivery of the PFA energy comprises inhibiting further delivery of the PFA energy responsive to the catheter distal end exiting the approved therapy area.

20. The method of claim 16, further comprising:
presenting an image with a proposed designated area of the heart muscle to an operator via a display; and
receiving user input increasing or decreasing the designated area relative to the proposed designated area.
